(19) United States
(12) Patent Application Publication
(10) Pub. No.: US 2017/0319222 A1
(43) Pub. Date: Nov. 9, 2017

(54) RETRIEVAL DEVICES, METHODS, AND SYSTEMS

(71) Applicant: Boston Scientific Scimed, Inc., Maple Grove, MN (US)

(72) Inventor: Michael S.H. Chu, Brookline, MA (US)

(73) Assignee: Boston Scientific Scimed, Inc., Maple Grove, MN (US)

(21) Appl. No.: 15/583,175

(22) Filed: May 1, 2017

Related U.S. Application Data
(60) Provisional application No. 62/332,140, filed on May 5, 2016.

(51) Int. Cl. A61B 17/221; A61B 17/00

(52) U.S. Cl. CPC A61B 17/221; A61B 2017/00862; A61B 2017/2212

(57) ABSTRACT

Aspects of retrieval devices, methods, and systems are disclosed. One aspect is a retrieval device comprising: a housing; a plunger with a distal end rotatably and slidably mounted in the housing; a sheath extending distally from the housing to define a sheath lumen with a sheath lumen opening; a drive wire extending distally from the plunger, through the housing, and into the sheath lumen; an end effector on a distal end of the drive wire; and a resilient element that biases the plunger proximally between a first position, wherein the end effector is proximal of the sheath lumen opening, and a second position, wherein at least a portion of the end effector is distal of the sheath lumen opening. Related methods and systems are also disclosed.

### CROSS-REFERENCE TO RELATED APPLICATIONS

This patent application claims the benefit of priority under 35 U.S.C. §119 to U.S. Provisional Patent Application No. 62/332,140, filed May 5, 2016, which is herein incorporated by reference in its entirety.

### TECHNICAL FIELD

Aspects of the present disclosure generally relate to medical devices and procedures. In particular, aspects relate to retrieval devices, methods, and systems.

### BACKGROUND

Retrieval devices are often used to remove organic material (e.g., blood clots, tissue, and biological concretions, such as urinary, biliary, and pancreatic stones) and inorganic material (e.g., components of a medical device or other foreign matter) from a body cavity. Without removal, such materials may obstruct the body cavity, block any passages leading thereto, and/or cause biological concretions to develop in certain parts of the body, such as in the kidneys, pancreas, ureter, and gallbladder. Minimally invasive medical procedures can remove these materials through a natural orifice or an incision, such as during a percutaneous nephrolithotomy (“PNCL”) procedure. Retrieval devices are also used in lithotripsy and ureteroscopy procedures to treat urinary calculi (e.g., kidney stones) in the ureter of a patient.

To reach the body cavity, some retrieval devices may include baskets passed through a working channel of an insertion device, such as an endoscope, ureteroscope, or laparoscope. For example, a distal opening of the working channel may be positioned in the body cavity. The retrieval device may be moved through the distal opening into the body cavity to retrieve the material. Two operators are typically required to operate the retrieval device: one operator to hold the insertion device, and another operator to move the retrieval device. Using multiple operators increases both the cost and complexity of the medical procedure. For example, each additional operator may add an incremental cost, and all operators must carefully coordinate their movements during the procedure.

The retrieval devices, methods, and systems described herein may rectify some of the deficiencies described above or address other aspects of the prior art.

### SUMMARY

Aspects of the present disclosure relate to retrieval devices, methods, and systems. Numerous aspects of the present disclosure are now described.

One aspect of this disclosure is a retrieval device. The device may comprise a housing; a plunger with a distal end rotatably and slidably mounted in the housing; a sheath extending distally from the housing to define a sheath lumen with a sheath lumen opening; a drive wire extending distally from the plunger, through the housing, and into the sheath lumen; an end effector on a distal end of the drive wire; and a resilient element that biases the plunger proximally between a first position, wherein the end effector is proximal of the sheath lumen opening, and a second position, wherein at least a portion of the end effector is distal of the sheath lumen opening.

According to this aspect, the end effector may be expanded when the plunger is in the second position. The entirety of the end effector may be distal of the sheath lumen opening when the plunger is in the second position. The end effector may self-expandable. For example, the end effector may be fully expanded when the plunger is in the second position. The end effector may be a basket, or a self-expandable basket, which may be made of a shape memory metal. The basket may comprise a distal end, a proximal end, and a plurality of wires extending therebetween to define a plurality of basket openings. The plurality of wires may be movable to change the size of one of the plurality of basket openings. The plunger may have a grip surface. The device may comprise at least one indicator on the plunger that is spaced apart from a proximal end of the housing by a distance equal to a stroke length of the plunger. The housing may further comprise an interface coupleable with an insertion device. The insertion device may have a working channel configured to receive the sheath. At least a portion of the end effector may be distal of a distal opening of the working channel when, for example, the sheath is received in working channel, the housing is coupled to the insertion device, and the plunger is in the second position.

In some aspects, the device further comprises an extension housing. The sheath may extend distally through the extension housing. The housing may be received in the extension housing for selective movement between a first locked position and at least one second locked position. For example, the housing may have a protrusion, the extension housing may have a track with a first lock tab and at least one second lock tab, and the housing may be moved by guiding the protrusion through the track and into one of the first or at least one second lock tabs.

Another aspect is a retrieval device. The device may comprise: a housing with an interface coupleable with an insertion device; a plunger with a distal end rotatably and slidably mounted in the housing; a lumen extending through the housing and the plunger to receive a drive wire; a resilient element that biases the plunger proximally between a first position, wherein the resilient element is expanded, and a second position, wherein the resilient element is compressed; and at least one indicator spaced apart from the housing to provide a measure of change for the resilient element.

According to this aspect, the device may further comprise a drive wire extending distally from the plunger, through the housing, and out of the interface. The device may include an end effector on the drive wire. A distance between the at least one indicator and a proximal end of the housing may be equal to a stroke length of the plunger. The housing may be received in an extension housing for selective movement between a plurality of locked positions. The plunger may be operable in each of the plurality of locked positions.

Another aspect is a method comprising: grasping a retrieval device including a housing, a plunger with a distal end rotatably and slidably mounted in the housing, a sheath extending distally from the housing to define a sheath lumen with a sheath lumen opening, a drive wire extending distally from the plunger, through the housing, and into the sheath lumen, an end effector on a distal end of the drive wire, and a resilient element that biases the plunger proximally between a first position, wherein the end effector is proximal of the sheath lumen opening, and a second position, wherein at least a portion of the end effector is distal of the sheath lumen opening; positioning the sheath lumen opening in a body cavity; moving the plunger towards the second position to extend the end effector towards a material in the body cavity; placing the material in the end effector; and moving the plunger towards the first position to capture the material in the end effector.

According to this aspect, placing the material in the end effector may include applying at least one of an axial force or a rotational force to the plunger. For example, placing the material in the end effector may comprise: positioning a portion of the end effector adjacent an internal surface of the body cavity; rotating the end effector against the internal surface to define an expanded opening in the end effector; and placing the material in the expanded opening. The method may further comprise: inserting the sheath into a working channel of an insertion device, the working channel having a distal opening, the sheath lumen opening being adjacent the distal opening of the working channel when inserted therein; and coupling the retrieval device to the insertion device so that the plunger is operable between a first position, wherein the end effector is proximal of the distal opening of the working channel, and a second position, wherein at least a portion of the end effector is distal of the distal opening of the working channel. Coupling the retrieval device to the insertion device may comprise coupling an extension housing of the retrieval device to an interface of the insertion device. The method may further comprise moving the housing in the extension housing between a first locked position, wherein the sheath lumen opening is proximal of the distal opening of the working channel, and a second locked position, wherein the sheath lumen opening is distal of the distal opening of the working channel.

It may be understood that both the foregoing summary and the following detailed descriptions are exemplary and explanatory only, neither being restrictive of the inventions claimed below.

### BRIEF DESCRIPTION OF THE DRAWINGS

The accompanying drawings are incorporated in and constitute a part of this specification. These drawings illustrate aspects of the present disclosure that, together with the written descriptions, serve to explain the principles of this disclosure.

FIG. 1A depicts a perspective view of an exemplary retrieval device according to the present disclosure.

FIG. 1B depicts a section view of the retrieval device of FIG. 1A.

FIG. 2A depicts a perspective view of the device of FIGS. 1A-B together with an exemplary drive wire including an exemplary end effector.

FIG. 2B depicts a section view of the device of FIG. 2A.

FIG. 3A depicts a perspective view of the device of FIGS. 2A-B together with an exemplary sheath, wherein the end effector is collapsed in the sheath.

FIG. 3B depicts a section view of the device of FIG. 3A, wherein the end effector is extended from the sheath.

FIG. 4 depicts a perspective view of an exemplary system according to the present disclosure, the system including the device of FIGS. 3A-B coupled to an exemplary insertion device.

FIG. 5A depicts an exemplary method of using the system of FIG. 4.

FIG. 5B depicts another exemplary method of using the system of FIG. 4.

FIG. 6A depicts a perspective view of another exemplary retrieval device coupled to an exemplary extension housing according to the present disclosure.

FIG. 6B depicts an enlarged view of the extension housing of FIG. 6A.

FIGS. 7A-B depict exemplary methods according to the present disclosure.

### DETAILED DESCRIPTION

Aspects of the present disclosure are now described with reference to exemplary retrieval devices, methods, and systems. Some aspects are described with reference to a lithotripsy procedure, wherein a distal end of a drive wire is positioned in a body cavity such as the interior of a kidney. During this procedure, an end effector on the drive wire, such as a basket, may be used to remove a material, such as a kidney stone, from the body cavity. Any reference to a particular procedure, such as lithotripsy; a particular material, such as a kidney stone or portion thereof; or a particular body cavity, such as the interior of a kidney, is provided for convenience and not intended to limit the present disclosure unless claimed. Accordingly, the concepts described herein may be utilized for any analogous device, method, or system—medical or otherwise.

The directional terms “proximal” and “distal,” and their respective initials “P” and “D,” are used to describe relative components and features of the present disclosure. Proximal refers to a position closer to the exterior of the body or a user, whereas distal refers to a position closer to the interior of the body or further away from the user. Appending the initials P or D to an element number signifies its proximal or distal location. Unless claimed, these directional terms and initials are provided for convenience and not intended to limit the present disclosure to a particular direction or orientation. As used herein, the terms “comprises,” “comprising,” or like variation, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.”

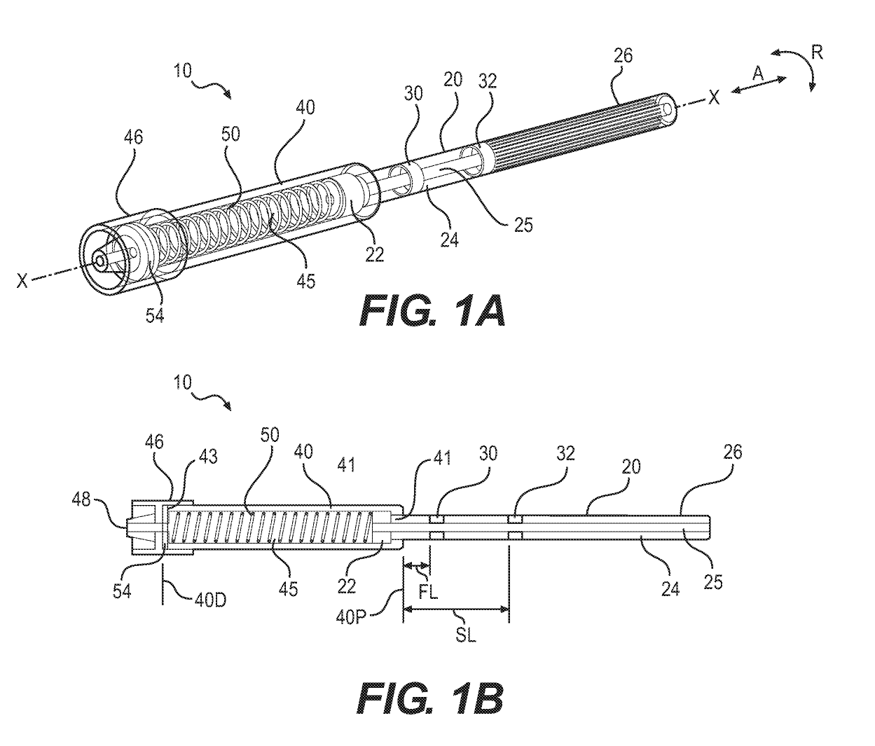

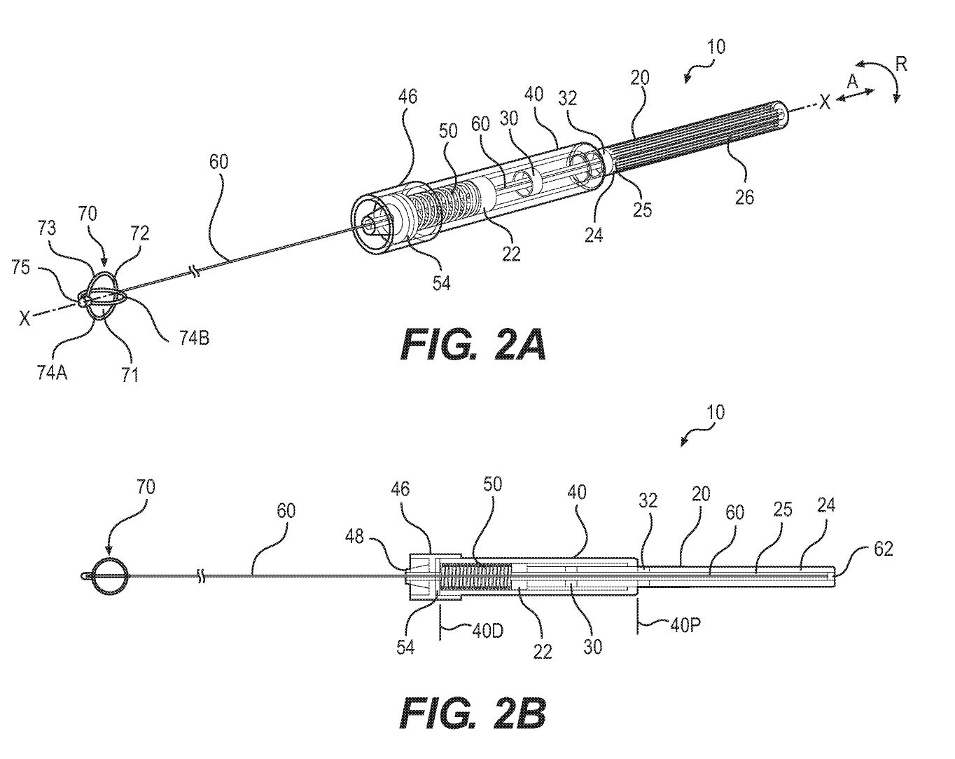

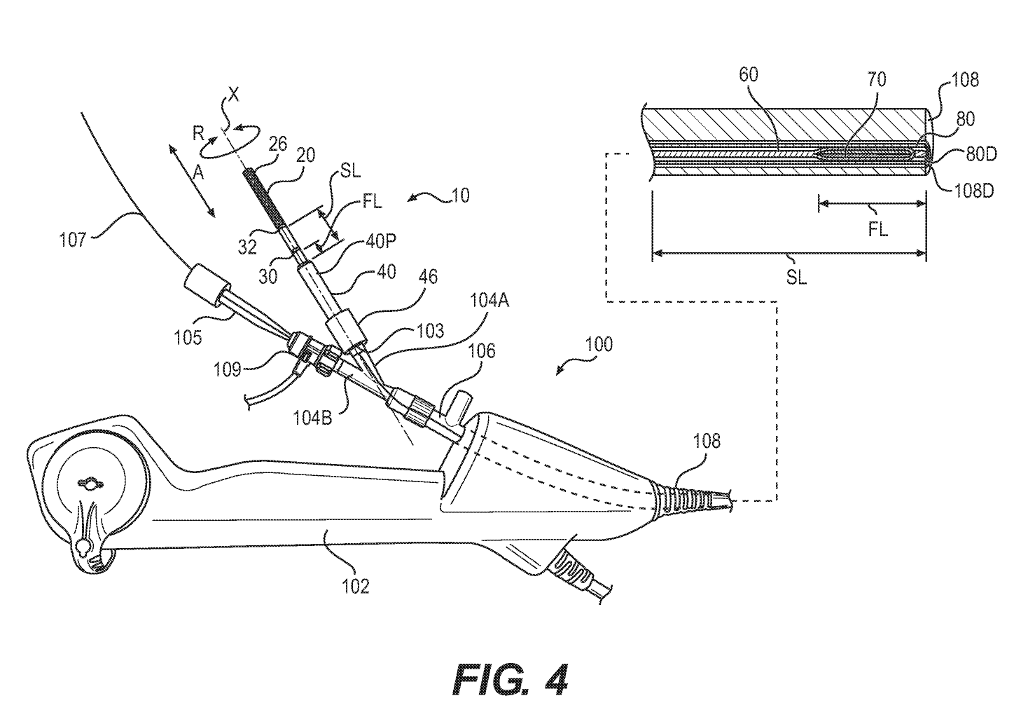

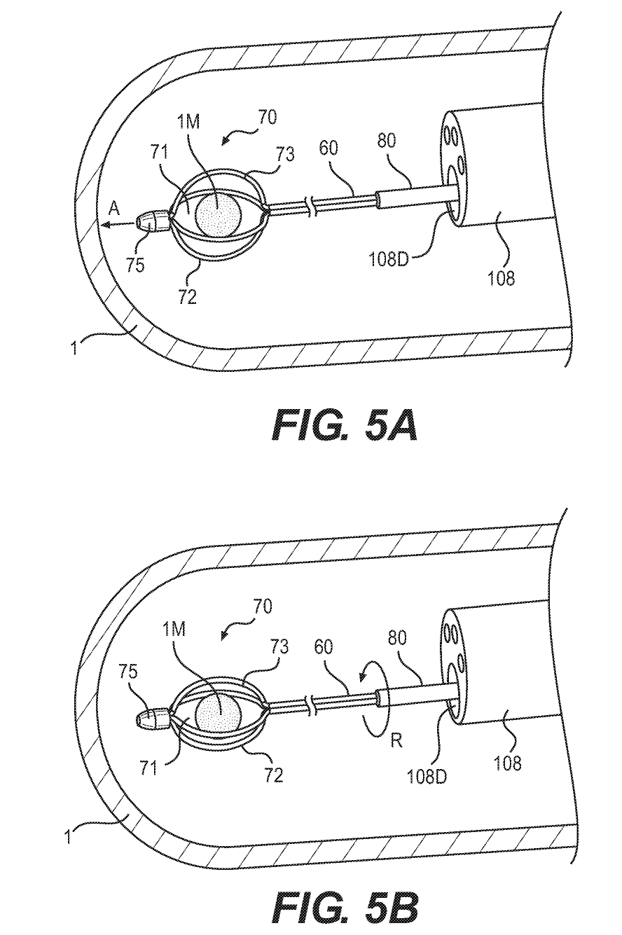

Aspects of the present disclosure describe a retrieval device 10 shown in FIGS. 1A-B. According to one aspect, retrieval device 10 comprises a plunger 20 mounted in a housing 40 for movement along a longitudinal axis X-X. Plunger 20 has a maximum length of movement or stroke length inside of housing 40 along axis X-X. Housing 40 may be coupled to an insertion device 102 having a working channel 108 (e.g., FIG. 4), such as a flexible ureteroscope. When coupled together, a handle portion of insertion device 102 may be held in one hand while device 10 is operated by the other hand to advance an end effector 70 (e.g., FIGS. 2A-B) out of channel 108 by moving plunger 20. This configuration may allow a single operator to perform, for example, a flexible ureterorenoscopy procedure, wherein a material 1M is removed from a body cavity 1 (e.g., FIGS. 5A-B). Elements of device 10 are now described.

Plunger 20 of FIGS. 1A-B includes a catch portion 22, a shaft portion 24, and a plunger lumen 25 extending therethrough. Catch portion 22 is located at a distal end of plunger 20. Shaft portion 24 has a diameter that is smaller than a diameter of catch portion 22. A grip surface 26 is positioned on the exterior surface of a proximal end of shaft portion 24. Grip surface 26 may include a plurality of raised elements that are grippable between, for example, a thumb and a finger on one hand of the operator. A first indicator 30 and a second indicator 32 are located on plunger 20 between catch portion 22 and grip surface 26, although any number of indicators may be provided on any position of plunger 20. In some aspects, plunger 20 is made of a polymeric material, portions of which may be opaque or transparent.

Housing 40 of FIGS. 1A-B includes a distal end 40D offset from a proximal end 40P to define an interior cavity 45 therebetween. Proximal end 40P has a shaft portion opening 41 with a diameter sized to receive shaft portion 24 of plunger 20, while distal end 40D has a catch portion opening 43 with a diameter sized to receive catch portion 22 of plunger 20. A diameter of interior cavity 45 is approximately equal to the diameter of catch portion opening 43. Accordingly, plunger 20 may be inserted proximally through housing 40 along longitudinal axis X-X until a proximal-facing surface of catch portion 22 is adjacent a distal-facing surface of housing 40, as in FIGS. 1A-B.

A resilient element 50 is depicted in FIGS. 1A-B as being placed in cavity 45. Element 50 may be a spring, which may made of metal or other resilient material. Interface 46 may be attached to distal end 40D of housing 40 to, for example, retain catch portion 22 of plunger 20 and resilient element 50 in interior cavity 45, thereby allowing plunger 20 to be mounted for movement in housing 40. Interface 46 is illustrated in FIGS. 1A-B as a male luer with an interface lumen 48. A diameter of interface lumen 48 is approximately equal to the diameter of plunger lumen 25 so that a drive wire, such as drive wire 60 of FIGS. 2A-B, may be inserted through the entirety of device 10 along axis X-X after plunger 20 has been mounted in housing 20.

In FIGS. 1A-B, a sealing element 54 is disposed between the proximal-facing surface of distal end 40D and a distal-facing surface of resilient element 50 to seal interior cavity 45. Sealing element 54 may be a seal (e.g., a wiper seal), a diaphragm, or like element made from a silicone, a low durometer rubber, or like material. In some aspects, element 54 has a continuous outer edge sealable against an interior surface of cavity 45, and a continuous inner edge sealable against an exterior surface of drive wire 60. In other aspects, an interior portion of element 54 may be a diaphragm that is pierced by wire 60 at is passes therethrough. Sealing element 54 may seal interior cavity 45 without additional accessories, and multiple sealing elements 54 may be used anywhere on device 10.

Once mounted in housing 40, plunger 20 may be moved along longitudinal axis X-X by application of an axial force A, and/or a rotated about axis X-X by application of rotational force R. Axial and rotational forces A and R may be applied to grip surface 26. The interior surfaces of shaft portion opening 41 and catch portion opening 43 may extend along a portion of axis X-X guide the movement of plunger 20 relative to housing 40. Resilient element 50 biases plunger 20 proximally along axis X-X by generating an axial response force that, when axial force A is released, moves plunger 20 proximally. Element 50 also may bias plunger 20 rotationally about axis X-X towards an initial position of alignment with housing 40. For example, element 50 may be a spring that is attached to plunger 20 and housing 40 and, thus, torqued when plunger 20 is rotated about axis X-X, under rotational force R, resulting in a rotational response force that, when rotational force R is released, returns plunger 20 to the initial position.

Resilient element 50 may define the stroke length of plunger 20. For example, plunger 20 may assume a first or extended position (e.g., FIG. 1A), wherein element 50 has an extended length and the proximal-facing surface of catch portion 22 is adjacent a distal facing surface of housing 40. Element 50 is compressed when plunger 20 moves distally along axis X-X. For example, plunger 20 may be moved distally from the first position towards a second or depressed position (e.g., FIGS. 2A-B), wherein element 50 has a compressed length equal to its solid height and the proximal-facing surface of catch portion 22 is remote from housing 40. Accordingly, the stroke length of plunger 20 may be defined as the distance between the first or second positions, or a difference of the extended and compressed lengths of element 50.

First indicator 30 and second indicator 32 of plunger 20 are configured to provide relativity between plunger 20 and housing 40. As shown in FIG. 1B, when plunger 20 is in the first position, first indicator 30 is spaced apart from the proximal end 40P of housing 40 by a first length FL, while second indicator 32 is spaced apart from proximal end 40P by a second length SL. Various relative measures may be obtained by moving plunger 20 between the first and second positions. For example, second length SL may be approximately equal to the stroke length of plunger 20 so that second indicator 32 is adjacent proximal end 40P when plunger 20 is in the second position (e.g., FIG. 2B). Additional relative measures may be established by comparing the distance between first indicator 30 and proximal end 40P, and/or the rotational position of indicators 30 or 32 relative to housing 40. These measures may, as described below, allow an operator to determine the size and/or position of material 1M.

Another aspect of the present disclosure is described with reference to FIGS. 2A-B, wherein a drive wire 60 is inserted into plunger lumen 25, through interior cavity 45, and out of interface lumen 48. Wire 60 may be approximately equal to or less than 1.3 French. A proximal portion 62 of wire 60 may be attached to plunger 20, using a chemical adhesive, a mechanical fit, a physical bonding, or like attachment means, any of which may be used to attach any two or more elements described herein. For example, proximal portion 62 of wire 60 in FIG. 2B may be adhered to plunger lumen 25 so that wire 60 is moveable with plunger 20 by application of axial or rotational forces A or R (e.g., FIG. 2A) to grip surface 26 of plunger 20.

End effector 70 may be disposed on the distal end of wire 60. End effector 70 may be a basket, a snare, a grasper, or the like, any of which may be made of shape memory metal. For example, end effector 70 may be made of a stainless steel, nickel titanium alloy, or other material that can be shaped, heated, or set to form a self-expandable shape when unrestrained by an external force. As shown in FIGS. 2A-B, end effector 70 may be, for example, a wire-form basket 72 including four wire segments 73 shaped to define four openings 71 therebetween. The exemplary basket 72 of FIGS. 2A-B may be formed by the intersection of a first wire-form shape 74A and a second wire-form shape 74B. A proximal portion of shapes 74A and 74B in FIGS. 2A-B may be attached to or formed integral with the distal end of wire 60, while a distal portion of shapes 74A and 74B is joined by a tip 75. Shapes 74A and 74B may, for example, be braided or weaved from wire 60. Tip 75 may be attached (e.g., welded) to both of shapes 74A and 74B to increase the rigidity of basket 72, or just one of shapes 74A or 74B to increase the flexibility of basket 72. In some aspects, basket 72 may be cut (e.g., laser cut) from a distal end portion of wire 60, such that each of shape 74A-B, wire segment 73, and/or tip 75 is integral with wire 60.

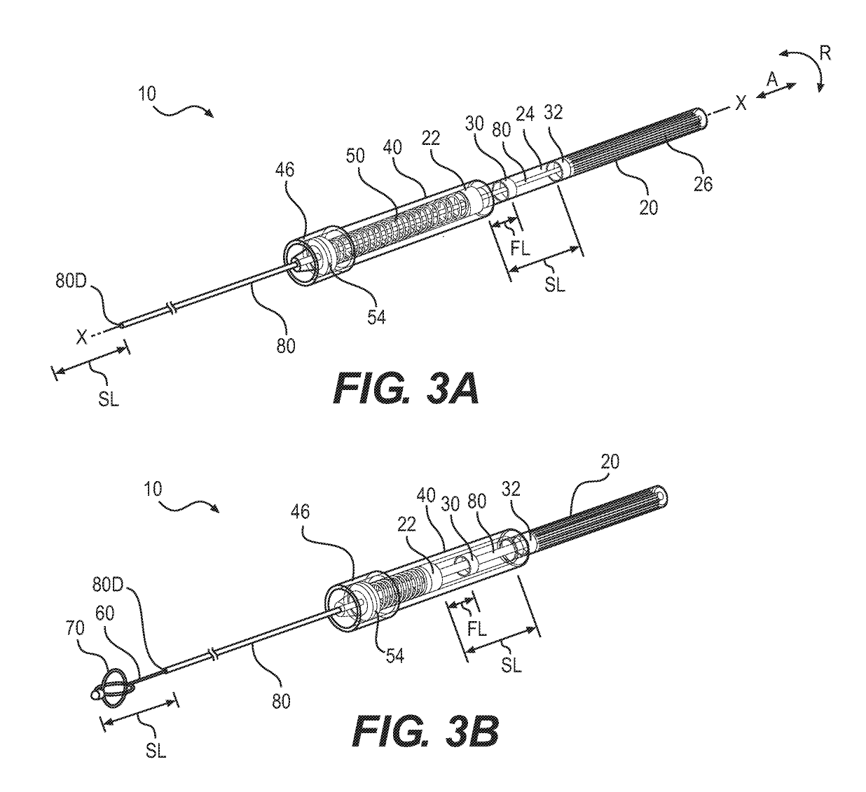

In some aspects, as in FIGS. 3A-B, a sheath 80 may be attached to interface 46 of housing 40. Drive wire 60 and/or end effector 70 may be housed within sheath 80. Wire 60 and sheath 80 may, together, be approximately equal to or less than 1.3 French. Because sheath 80 is attached to interface 46, axial force A may be used to move plunger 20 from the first position, wherein end-effector 70 is proximal of a distal opening 80D of sheath 80 (e.g., FIG. 3A), to the second position, wherein at least a portion of end-effector 70 is distal of distal opening 80D of sheath 80 (FIG. 3B). For example, basket 72 of FIGS. 2A-B may be collapsed into a closed state within sheath 80 (e.g., FIG. 3A) when plunger 20 is in the first position, and expanded into an open state (e.g., FIG. 3B) when plunger 20 is the second position. A distal portion of sheath 80 may be used to maintain end-effector 70 in the closed state, and/or move end-effector 70 into the closed state. The interior edges of distal openings 80D, for example, may be configured to collapse end-effector 70 into the closed state, collapse end effector around material 1M, and/or capture material 1M (e.g., FIGS. 5A-B), either in end-effector 70, in distal opening 80D, and/or against the distal face of sheath 80.

The first and second indicators 30 and 32 of plunger 20 may be configured for use with wire 60, end effector 70, and sheath 80. In some aspects, second length SL is equal to the stroke length of plunger 20, while first length FL is equal to a distance relative to end effector 70. For example, end effector 70 may be configure to capture material 1M and retract it through an access sheath having an inner diameter of, for example, 11 French. First length FL may be defined with respect to this inner diameter by placing a spherical ball having a diameter equal to 11 French, and allowing end effector 70 to close around the spherical ball. First indicator 30 is placed adjacent proximal end 40P once the spherical ball has been captured in end effector 70, thereby defining first length FL to be approximately equal to a length of end effector 70 surrounding material 1M. Accordingly, if material 1M has a dimension greater is captured in end effector 70, and first indicator 30 is distal of proximal end 40P, e.g., inside of housing 40, then material 1M cannot be retracted through the access sheath; whereas if first indicator 30 is proximal of proximal end 40P, then material 1M can be retracted through the access sheath.

In some aspects, multiple first indicators 30 may be provided. For example, device 10 may be configured for use with multiple access sheaths, each having a different inner diameter. Three access sheaths having respective inner diameters of 11 French, 12 French, and 13 French may be used. In this example, three first indicators 30 may be provided, each defining a first length FL with respect to one of these three inner diameters, with the 11 French indicator 30 being distal of the 12 French indicator 30, and the 12 French indicator being distal of the 13 French indicator 30. An approximate size of material 1M can be made by viewing the respective 11, 12, and 13 French indicators 30. Specifically, if material 1M is captured in end effector 70 and none of the 11, 12, or 13 French indicators 30 are proximal of proximal end 40P of housing 40, then material 1M cannot be retracted through any of the access sheaths. If only the 13 French indicator 30 is proximal of proximal end 40P, then material 1M can only be retracted through the 13 French access sheath. If the 12 and 13 French indicators 30 are proximal of proximal end 40P, then material 1M can be retracted through either of the 12 or 13 French access sheaths. And if all three of the 11, 12, and 13 French indicators 30 are proximal of proximal end 40P, then material 1M can be retracted through any of the 11, 12, or 13 French access sheaths.

Another aspect of the present disclosure is described with reference to a system 100 of FIG. 4. As shown, system 100 may include device 10 and insertion device 102 (e.g., a LithoVue™ Single-Use Digital Ureteroscope by Boston Scientific). Interface 46 (e.g., a male luer) of device 10 may be coupled to an interface 103 (e.g., a female luer) of insertion device 102. Interface 103 is depicted, for example, as being attached to a Y-shaped connector including a first branch 104A and a second branch 104B. Each of branches 104A and 104B have a lumen extending therethrough to be in communication with a port 106 of device 102. As shown, the working channel 108 of insertion device 102 extends between port 106 and distal opening 108D. Channel 108 may be approximately 3.6 French. In FIG. 4, interface 103 is attached to branch 104A, while a source connection 109 (e.g., a Urolock™ Adaptor by Boston Scientific) and an introducer 105 are attached to branch 104B.

In FIG. 4 and FIGS. 5A-B, sheath 80 and drive wire 60 extend into working channel 108 through first branch 104A. Sheath 80 may be received in working channel 108 so that the distal opening 80D of sheath 80 is adjacent the distal opening 108D of working channel 108 when interface 46 is coupled to interface 103. Opening 80D may be proximal, adjacent or distal of opening 108D when interfaces 46 and 103 are coupled together. Accordingly, in system 100, axial force A may move plunger 20 from the first position, wherein end-effector 70 is proximal of distal opening 108D of working channel 108 and/or distal opening 80D of sheath 80, to the second position, wherein at least a portion of end-effector 70 is distal of openings 108D and/or 80D. As before, a distal portion of sheath 80 and/or working channel 108, such as the interior edges of openings 80D and/or 108D, may be used to maintain end-effector 70 in the closed state, and/or move end-effector 70 into the closed state.

The angle of first branch 104A relative to second branch 104B allows a second device to be placed in working channel 108. As shown in FIG. 4, for example, a laser fiber 107 may extend through second branch 104B into working channel 108 with end effector 70. Laser energy may be emitted from fiber 107 onto material 1M from a laser source to, for example, reduce the size of material 1M. Source connection 109 is configured to deliver fluid or aspiration for working channel 108. A T-connector is depicted on port 106 in FIG. 4 to provide another means for fluid management.

Resilient element 50 (e.g., FIGS. 1A-3B) may be configured for use in system 100. For example, element 50 may be configured to apply an axial restoring force sufficient to capture material 1M without damaging the respective distal openings 80D and 108D of sheath 80 and/or working channel 108. This may be particularly important if sheath 80 and/or channel 108 are made from disposable materials, such as polymers, wherein element 50 may be optimized to limit the stress and strain applied to such materials by the interaction of end effector 70 and/or material 1M with element 50.

First and second indicators 30 and 32 of plunger 20 also may be configured for use in system 100. First length FL may determine the size of material 1M, and whether it will fit into sheath 80, working channel 108 and/or an access sheath or anatomical passageway used to pass sheath 80 and/or channel 108 into body cavity 1. For example, first length FL may allow an operator to determine whether material 1M can be retracted through an access sheath, as described above; or whether laser energy from laser fiber 107 should be used to reduce the size of material 1M before attempting to retract it through the access sheath. Second length SL may, as before, approximately equal to the stroke length of plunger 20.

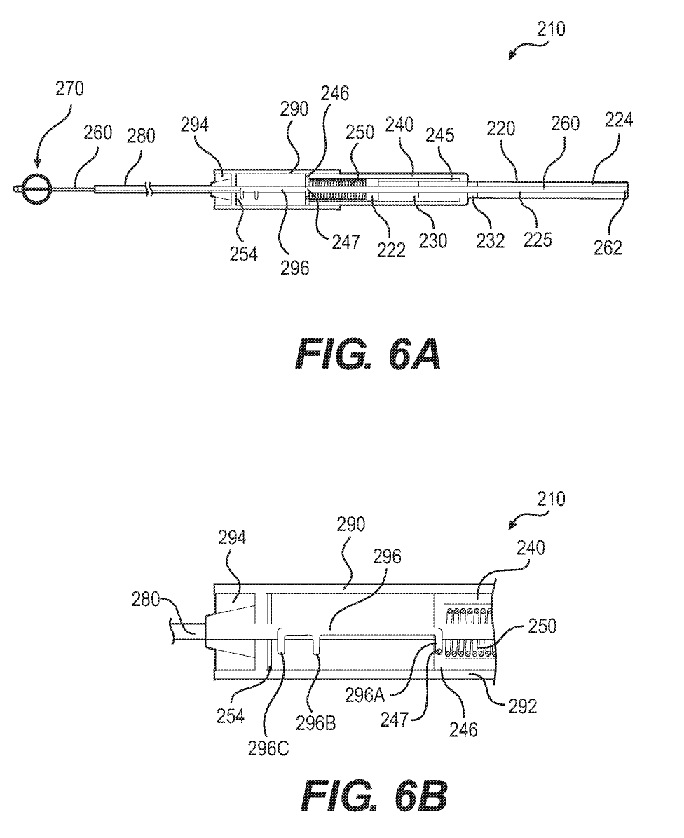

Another aspect of the present disclosure is described with reference to a device 210 of FIGS. 6A-B. Device 210 is similar to device 10, except that interface 46 has been modified for use with an extension housing 290. For example, interface 246 of housing 240 includes at least one protrusion 247 extending outwardly therefrom to engage extension housing 290. Accordingly, like element numbers are used to described the like elements of devices 10 and 210. As shown in FIGS. 6A-B, device 210 is coupled to extension housing 290 and includes a drive wire 260, an end effector 270, and a sheath 280. Extension housing extension 290 increases the range of movement for housing 240 and, thus, the reach of end effector 270.

Extension housing 290 includes a proximal interface 292, a distal interface 294, a track 296, and a seal 254. Distal interface 294 may be coupled to interface 103 of device 102 (e.g., FIG. 4). For example, distal interface 294 may be a male luer, similar to interface 46 of device 10 (e.g., FIG. 1). Housing 240 is receivable in proximal interface 292 so that plunger 220 and housing 240 may move independently of extension housing 290. Protrusion 247 of housing 240 is positioned in track 296 when housing 240 is received in proximal interface 292. As shown in FIGS. 6A-B, track 296 has an proximal position 296A, an intermediate position 296B, and a distal position 296C. Each of positions 296A-C is contained in an offshoot or lock tab extending away from a linear portion of track 296. Protrusion 247 may be at position 296A when the distal end of housing 240 is received in interface 292. Any number of such positions may be provided on track 296, in any geometric configuration. For example, extension housing 290 may be modified so that positions 296A-C are arranged in a stair-like configuration. One protrusion 247 may extend from each side of interface 246 for receipt in a track 296 on each side of housing 240. Alternatively, housing 240 and extension housing 290 may be further modified to include alternate movement mechanisms, such as a corresponding set of threads, a rack and pinion arrangement, or a ratchet assembly, any of which may allow housing 240 to move relative to extension housing 290.

These configurations allow sheath 280 to move along axis X-X. For example, distal interface 294 of extension housing 209 may be coupled to interface 103 of insertion device 102 so that a distal opening 280D of sheath 280 is proximal of distal opening 108D of working channel 108 when protrusion 247 is received in proximal position 296A. A set of axial and rotational forces, similar to forces A and R, may be applied to housing 240 to move protrusion 247 from proximal position 296A into the intermediate and/or distal positions 296B, 296C. Distal opening 280D of sheath 280 may be adjacent distal opening 108D of working channel 108 when protrusion 247 is at intermediate position 296B. Opening 280D may be distal of opening 108D when protrusion 247 is at distal position 296C. Arranging positions 296B and 296C in lock tabs allows protrusion 247 to lock the position of housing 240 relative to extension housing 290. Other locking means may be provided. For example, each lock tab may be a narrowed portion of track 296. Plunger 220 may be used to operate end effector 270 in all such positions.

Device 210 may used with housing extension 290, with or without sheath 280. For example, the length of drive wire 260 may correspond with a length of working channel 108 so that end effector 270 is contained in channel 108 when protrusion 247 is at position 296A, extended a first distance beyond distal end 108D at position 296B, and extended a second distance beyond distal end 108D at position 296C. Protrusion 247 may be moved to position 296A in order to capture material 1M.

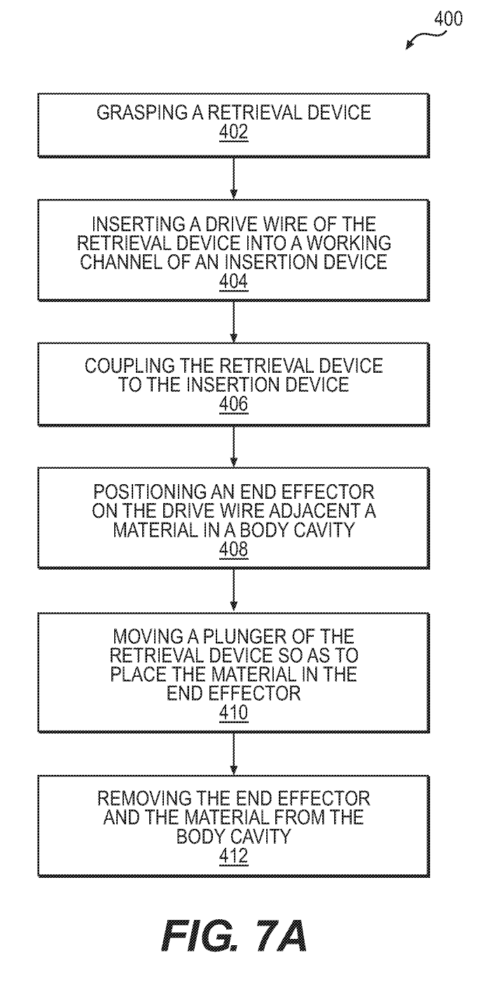

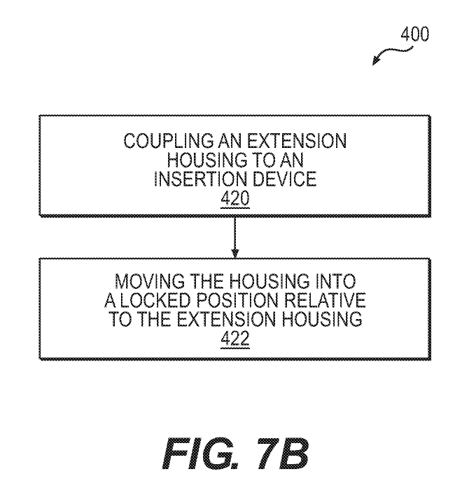

Aspects of a method 400 are now described with reference to device 10, device 210, and system 100. Method 400 is depicted in FIG. 7A and may comprise a step 402 of grasping device 10, a step 408 of positioning end effector 70 adjacent a material in body cavity 1, and a step 410 for moving plunger 20 to place material 1M in end effector 70 (e.g., FIGS. 5A-B). Device 10 may be grasped by one hand of an operator. Axial force A and/or rotational force R may be applied to plunger 20 in step 410. For example, grip portion 26 of plunger 20 may be grasped between a thumb and finger of said one hand for application of forces A and R thereto. To retrieve material 1M, method 400 may further comprise a step 412 for removing end effector 70 and material 1M from body cavity 1.

Although not required, intermediate method steps may comprise a step 404 for inserting wire 60 into working channel 108 of insertion device 102 (e.g., FIG. 4), and a step 406 for coupling retrieval device 10 to insertion device 102. For example, devices 10 and 102 may be coupled such that resilient element 50 biases plunger 20 proximally between the first position (e.g., FIG. 4), wherein the end effector 70 is proximal of distal opening 108D of working channel 108, and the second position (e.g., FIGS. 5A-B), wherein at least a portion of end effector 70 is distal of opening 108D. Insertion device 102 may be grasped in one hand of the operator, while retrieval device 10 is grasped in the other. Step 408 may further comprise positioning distal opening 108D adjacent a material 1M in body cavity 1. For example, working channel 108 may be passed into body cavity 1 through the access channel described above. Step 410 may further comprise moving plunger 20 distally towards the second position to extend end effector 70, while step 412 further comprises moving plunger 20 proximally towards the first position to capture material 1M in end effector 70.

Method 400 may be further modified. For example, sheath 80 may be inserted into working channel 108 in step 404, and device 10 and 102 may be coupled to device 102 in step 406, such that resilient element 50 biases plunger 20 proximally between the first position, wherein the end effector 70 is proximal of distal opening 80D of sheath 80 (e.g., FIG. 3A) and/or distal opening 108 of working channel 108 (e.g., FIG. 4), and the second position, wherein at least a portion of end effector 70 is distal of opening 80D (e.g., FIG. 3B) and/or opening 108D (e.g., FIGS. 5A-B). Sheath 80 may be inserted into working channel 108 so that distal opening 80D is adjacent distal opening 108D. Plunger 20 may, thus, be operable (e.g., by a single hand) to move end effector 70 between the closed and open states described herein.

As noted above, axial force A and/or rotational force R may be applied to plunger 20 in step 410. Examples are now described with reference to end effector 70 of FIGS. 5A-B, which, as described above, may be a self-expandable basket 72 comprised of four segments 73 arranged to define four openings 71. In one example, step 410 may further comprise positioning an opening 71 of end effector 70 adjacent material 1M, and applying axial force A to plunger 20 in order to place material 1M into basket 72 by moving (e.g., flexing) one or more segments 73 until the opening 71 is large enough to receive material 1M (e.g., FIG. 5A). In another example, step 410 may comprise positioning an opening 71 adjacent material 1M such that at least one segment 73 is placed in contact with an interior surface of the body cavity 1, and using rotational force R to “spin scoop” material 1M into basket 72 by moving (e.g., rotating) at least one segment 73 against the interior surface and towards another segment 73 until the opening 71 is large enough to receive material 1M (e.g., FIG. 5B). In either example, when axial force A or rotational force R is released, then the resiliency of basket 72, and/or the responsive forces applied by resilient element 50, may aid in capturing material 1M.

Still other method steps may comprise determining, with first indicator 30, a size of material 1M, and whether it can be retracted through sheath 80, working channel 108, and/or an access sheath or anatomical passageway. Other steps may be used to locate on or more first indicators 30 on plunger 20, such as those using the spherical ball described above. If needed, another step may comprise using laser fiber 107 to reduce the size of material 1M before removing end effector 70 and material 1M from body cavity 1 in step 412. Still other steps may comprise determining, with reference to first and/or second indicators 30, 32, whether end effector 70 in an open or partially open state before, for example, performing step 410.

Additional method steps may be used with device 210 and extension housing 290 of FIGS. 6A-B. For example, as shown in FIG. 7B, method 400 may further comprise a step 420 for coupling distal interface 294 of extension housing 290 with interface 103 of device 102 (e.g., FIG. 4). Another step 422 may comprise moving housing 240 into one or more locked positions relative to extension housing 290. For example, housing 240 may be moved in step 422 from a first locked position, wherein distal opening 280D of sheath 280 is proximal of distal opening 108D of working channel 108, to a second locked position, wherein the distal end of sheath 280 is distal of opening 108D. Step 422 may further comprise any of placing protrusion 247 in proximal position 296A of track 296; moving housing 240 into the first locked position by moving protrusion 247 into intermediate position 296B; and/or moving housing 240 into the second locked position by moving protrusion 247 into distal position 296C. Extension housing 290 may be pre-assembled with housing 240. Alternatively, method 400 may include an additional step for receiving the distal end of housing 240 in extension housing 290 for movement between a plurality of positions. For example, extension housing 290 may coupled to insertion device 102 so that housing 240 may, in turn, be received in proximal interface 292 of extension housing 290.

While principles of the present disclosure are described herein with reference to illustrative aspects for particular applications, the disclosure is not limited thereto. Those having ordinary skill in the art and access to the teachings provided herein will recognize additional modifications, applications, aspects, and substitution of equivalents all fall in the scope of the aspects described herein. Accordingly, the present disclosure is not to be considered as limited by the foregoing description.

1. A retrieval device comprising:
a housing;
a plunger with a distal end rotatably and slidably mounted in the housing;
a sheath extending distally from the housing to define a sheath lumen with a sheath lumen opening;
a drive wire extending distally from the plunger, through the housing, and into the sheath lumen;
an end effector on a distal end of the drive wire; and
a resilient element that biases the plunger proximally between a first position, wherein the end effector is proximal of the sheath lumen opening, and a second position, wherein at least a portion of the end effector is distal of the sheath lumen opening.

2. The device of claim 1, wherein the end effector is fully expanded when the plunger is in the second position.

3. The device of claim 2, wherein the end effector is a self-expandable basket.

4. The device of claim 3, wherein the basket comprises a distal end, a proximal end, and a plurality of wires extending therebetween to define a plurality of basket openings.

5. The device of claim 4, wherein the plurality of wires are movable to change the size of one of the plurality of basket openings.

6. The device of claim 1, wherein the plunger has a grip surface.

7. The device of claim 1, further comprising at least one indicator on the plunger that is spaced apart from a proximal end of the housing by a distance equal to a stroke length of the plunger.

8. The device of claim 1, further comprising an extension housing, wherein the sheath extends distally through the extension housing, and the housing is mounted in the extension housing for selective movement between a first locked position and at least one second locked position distal of the first locked position.

9. The device of claim 8, wherein the housing has a protrusion, the extension housing has a track with a first lock tab and at least one second lock tab, and the housing is moved by guiding the protrusion through the track and into the respective first or at least one second lock tabs.

10. A retrieval device including;
a housing with an interface portion engageable with an insertion device;
a plunger with a distal end rotatably and slidably mounted in the housing;
a lumen extending through the housing and the plunger to receive a drive wire;
a resilient element that biases the plunger proximally between a first position, wherein the resilient element is expanded, and a second position, wherein the resilient element is compressed; and
at least one indicator spaced apart from the housing to provide a measure of change for the resilient element.

11. The device of claim 10, further comprising a drive wire extending distally from the plunger, through the housing, and out of the interface portion.

12. The device of claim 11, further comprising an end effector on the drive wire.

13. The device of claim 12, wherein a distance between the at least one indicator and a proximal end of the housing is equal to a stroke length of the plunger.

14. The device of claim 10, wherein the housing is mounted in an extension housing for selective movement between a plurality of locked positions, and the plunger is operable in each of the plurality of locked positions.

15. A method comprising:
grasping a retrieval device including;
a housing;
a plunger with a distal end rotatably and slidably mounted in the housing;
a sheath extending distally from the housing to define a sheath lumen with a sheath lumen opening;
a drive wire extending distally from the plunger, through the housing, and into the sheath lumen;
an end effector on a distal end of the drive wire; and
a resilient element that biases the plunger proximally between a first position, wherein the end effector is proximal of the sheath lumen opening, and a second position, wherein at least a portion of the end effector is distal of the sheath lumen opening;
positioning the sheath lumen opening in a body cavity;
moving the plunger towards the second position to extend the end effector towards a material in the body cavity;
placing the material in the end effector; and
moving the plunger towards the first position to capture the material in the end effector.

16. The method of claim 15, wherein placing the material in the end effector includes applying at least one of an axial force or a rotational force to the plunger.

17. The method of claim 15, wherein placing the material in the end effector comprises:
positioning a portion of the end effector adjacent an internal surface of the body cavity;
rotating the end effector against the internal surface to define an expanded opening in the end effector; and
placing the material in the expanded opening.

18. The method of claim 15, further comprising:
inserting the sheath into a working channel of an insertion device, the working channel having a distal opening, the sheath lumen opening being adjacent the distal opening of the working channel when inserted therein; and
coupling the retrieval device to the insertion device so that the plunger is operable between a first position, wherein the end effector is proximal of the distal opening of the working channel, and a second position, wherein at least a portion of the end effector is distal of the distal opening of the working channel.

19. The method of claim 18, wherein coupling the retrieval device to the insertion device comprises coupling an extension housing of the retrieval device to an interface of the insertion device.

20. The method of claim 19, further comprising moving the housing in the extension housing between a first locked position, wherein the sheath lumen opening is proximal of the distal opening of the working channel, and a second locked position, wherein the sheath lumen opening is distal of the distal opening of the working channel.
